(19) United States
(12) Patent Application Publication
(10) Pub. No.: US 2021/0113108 A1
(43) Pub. Date: Apr. 22, 2021

(54) METHOD, SYSTEM AND DEVICE FOR GENERATING ELECTROCARDIOGRAM WITH FEWER NUMBER OF PROBES

(71) Applicant: Sensesemi Technologies Private Limited, Bengaluru (IN)

(72) Inventors: Veena S. Chakravarthi, Bengaluru (IN); Vijaykumar Muktamath, Bengaluru (IN); Vishweshwara Mundkur, Bengaluru (IN); Suhasini S, Bengaluru (IN)

(73) Assignee: Sensesemi Technologies Private Limited, Bengaluru (IN)

(21) Appl. No.: 16/720,000

(22) Filed: Dec. 19, 2019

(30) Foreign Application Priority Data
Oct. 22, 2019 (IN) 201941042812

(51) Int. Cl. A61B 5/0408; A61B 5/0404; A61B 5/00

(52) U.S. Cl. CPC A61B 5/04085; A61B 5/72; A61B 5/0404

(57) ABSTRACT

According to an aspect of the present disclosure, a hand held device is providing twelve lead ECG signals using fewer number of probes/electrodes comprising, a first electrode to sense electrical RH signal, a second electrode to sense electrical LH signal, a third electrode to sense electrical LL signal, a fourth electrode to sense electrical V1 through V6 signal in a sequence, a first electronic circuit operative generate a lead I, lead II and lead III ECG signal, a second electronic circuit to generate a CT signal from the lead I, lead II and lead III signal and a third electronic circuit operative to generate V1-V6 lead ECG signals from the electrical V1 through V6 signal and the CT signal.

### BACKGROUND

### CROSS REFERENCES TO RELATED APPLICATIONS

This application claims priority from Indian patent application No. 201941042812 filed on Oct. 22, 2019 which is incorporated herein in its entirety by reference.

### TECHNICAL FIELD

Embodiments of the present disclosure relate generally to biomedical electronic devices and more particularly to method, system and device for generating electrocardiogram with fewer number of probes

### RELATED ART

Electro cardio gram (ECG) also referred to as EKG is a recording of electrical activity of a human heart. As is well known, several clinical devices and apparatus are employed to measure the electrical activity of the heart and represent the measured electrical signal in the form of ECG. The conventional clinical ECG devices and systems employ number of probes to measure electrical activity and provide twelve leads ECG signals.

The 12 leads of the ECG are recognized as lead I, II, III, aVR, aVL, aVF, V1, V2, V3, V4, V5, and V6 as is well known in the art. Details of generating conventional twelve leads ECG signals from probes (also referred to as Electrodes) and position of the probes on the human body etc., are more fully described in a book titled “Bioelectromagnetism—Principles and Applications of Bioelectric and Biomagnetic Fields”, authored by Jaakko Malmivuo and Robert Plonsey, published by Oxford University Press, which is incorporated herein by reference. Briefly, the 10 probes/electrodes are provided for measuring electrical activity of the heart.

The 10 probes are classified as 4 limb (or extremities) electrodes and 6 Precardial (or chest) electrodes. The four limb electrodes are Right Arm (RA), Left Arm (LA), left Leg (LL) and Right Leg (RL). In some cases, the RL probe on the right leg is connected/considered as reference electrode (reference potential) thereby requiring only 9 probes for measuring the electrical signals. Electrical signal measured by RA, LA and LL probes are used to capture signal and derive leads I, II, III, aVR, aVL, and aVF signals. Further, an additional reference potential referred to as Wilson central terminal (CT) is formed by connecting a resistance to each limb electrode and interconnecting the free end of the wires to form the CT common point. The CT common point or (Wilson central terminal) represents an average of the limb potentials aVR, aVL, and aVF. The 6 precardial probes are positioned/placed on the chest around the heart in a known way. The electrical signal measured by the 6 precardial probes is used to provide six leads (V1-V6) ECG signals. As may be seen, in order to provide all twelve leads signals (I, II, III, aVR, aVL, aVF, and V1-V6),the 10 electrodes (LA, RA, RL, LL, and V1-V6)need to be placed at the appropriate positions. Since these are physically at distant locations, 10 separate cables are usually employed making it cumbersome and complicated procedure at least for preliminary diagnostics. SUMMARY

According to an aspect of the present disclosure, a hand held device is providing twelve lead ECG signals using fewer number of probes/electrodes comprising, a first electrode to sense electrical RH signal, a second electrode to sense electrical LH signal, a third electrode to sense electrical LL signal, a fourth electrode to sense electrical V1 through V6 signal in a sequence, a first electronic circuit operative generate a lead I, lead II and lead III ECG signal, a second electronic circuit to generate a CT signal from the lead I, lead II and lead III signal and a third electronic circuit operative to generate V1-V6 lead ECG signals from the electrical V1 through V6 signal and the CT signal.

Several aspects are described below, with reference to diagrams. It should be understood that numerous specific details, relationships, and methods are set forth to provide a full understanding of the present disclosure. One who skilled in the relevant art, however, will readily recognize that the present disclosure can be practiced without one or more of the specific details, or with other methods, etc. In other instances, well-known structures or operations are not shown in detail to avoid obscuring the features of the present disclosure.

### BRIEF DESCRIPTION OF DRAWINGS

FIG. 1 is an example device providing twelve lead ECG signal using fewer number of probes/electrodes in an embodiment.

FIG. 2 is a block diagram illustrating an example generation of twelve leads ECG signals from four electrodes.

FIG. 3A is a block diagram illustrating the manner in which the leads I and II ECG signals are generated.

FIG. 3B is a block diagram illustrating the manner in which CT signal is generated in one embodiment.

FIG. 4is a block diagram illustrating the manner in which lead V1-V6 signals are generated in one embodiment.

FIG. 5 is a block diagram of a hand held device generating twelve lead ECG signals in one embodiment.

### DETAILED DESCRIPTION OF THE PREFERRED EXAMPLES

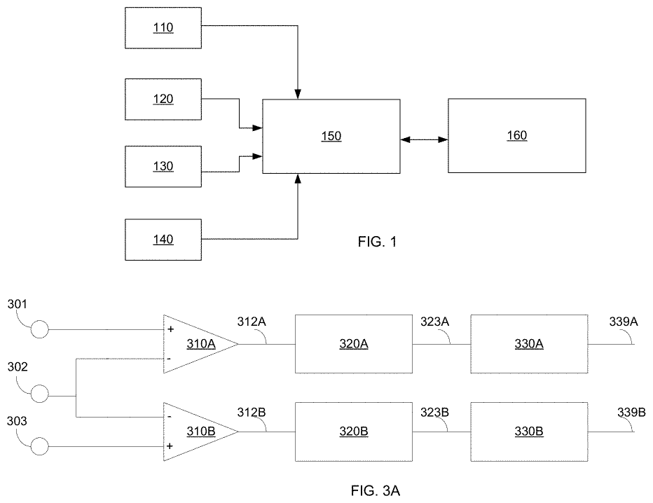

FIG. 1 is an example device providing twelve lead ECG signal using fewer number of probes/electrodes in an embodiment. The device 101 is shown comprising probes (electrodes) 110, 120, 130, and 140, 12 lead ECG generator 150, and output interface 160. Each element is described in further detail below.

The probes 110, 120, and 130are operative as limb electrodes to sense electrical signals on LA, RA and LL. In one embodiment, the 110, 120, and 130 probes are configured to receive electrical signal through one finger on the left arm, one finger on the right arm and upper part of the left leg respectively. Due to such configuration, the three probes 110, 120, and 130 may be mounted close to each other as fingers on both hands may easily reach the upper left leg part.

The probe 140 is flexible or movable probe that may be placed at multiple positions. In that, the probe 140 is configured to be placed over the chest at the positions corresponding to the leads V1 through V6 of the twelve leads ECG signals. Thus, probe 140 operates as precardial electrode. Accordingly, the probe 140 collects electrical signals for leads V1 through V6. In one embodiment, the probe 140 comprises single probe/electrode configured to place at positions V1 through V6 in a sequential manner. Thus, the probe 140 senses anyone of V1 through V6 at a given point in time. Alternatively, the probe 140 may comprise a set of electrodes in the form of a patch and may sense more than one of V1 through V6 leads simultaneously.

The 12 lead ECG generator 150 generates twelve leads ECG signals I, II, III, aVR, aVL, aVF, and V1 through V6 from the electrical signal captured by the probes 110, 120, 130, and 140. The twelve leads ECG signals are provided to the output interface 160. The output interface 160 may comprise plotter, electronic display device, memory and other electronic system, for example, configured to plot/display/store/apply the 12 leads ECG signals. The manner in which the 12 lead ECG generators 150 generates twelve leads ECG signal from the fewer probes (110, 120, 130, 140 for example) is further described below.

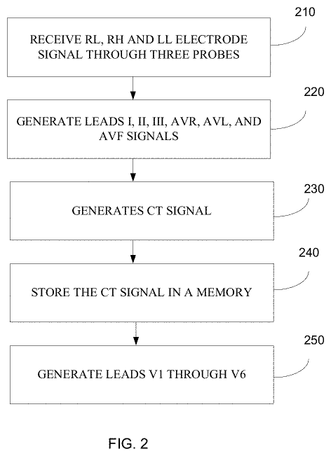

FIG. 2 is a block diagram illustrating a generation of twelve leads ECG signals from four electrodes. In block 210, the 12 lead ECG generator receives RL, RH and LL electrode signal through three probes. In block 220, the 12 lead ECG generator generates leads I, II, III, aVR, aVL, and aVF signals.

In block 230, the 12 lead ECG generator generates CT signal. In one embodiment, the CT signal is determined as an average of the potential measured by probes 110, 120, 130. In block 240, the 12 lead ECG generator, stores the CT signal in a memory. In block 250, the 12 lead ECG generator generates leads V1 through V6 signals as difference between the potential measured by the probe 140 and stored CT signal. In one embodiment, the leads V1 through V6 signals are generated sequentially when the probe 140 is sequentially placed over positions corresponding to V1 through V6 on the chest. As a result, the twelve lead ECG signals are generated without having to hold more than 3 probes at any given time. This enables a user to generate twelve lead ECG signals by self. The manner in which the 12 lead ECG generator may be implemented is further described below.

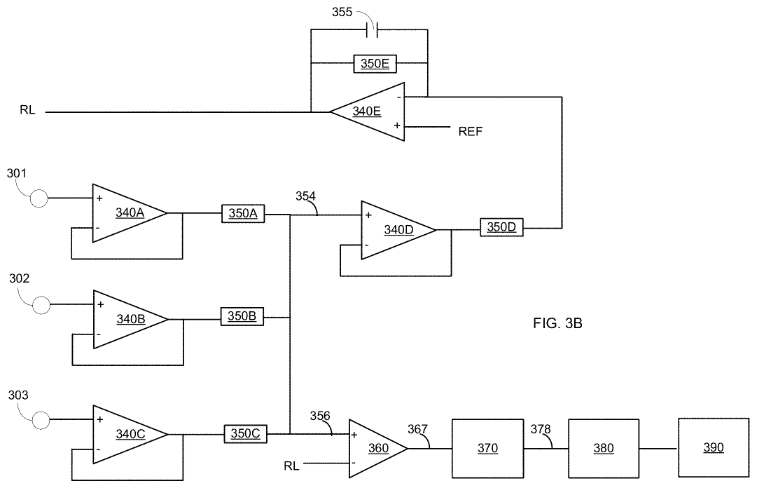

FIG. 3A is a block diagram illustrating the manner in which the leads I and II ECG signals are generated. The block diagram is shown comprising differential amplifiers 310A and 310B, analog to digital convertors (ADC) 320A and 320B, and digital filters 330A and 330B. In the block diagram, probe 301 sensing LA is coupled to the non inverting terminal and the probe 302 sensing RA is coupled to the inverting terminal of the differential amplifier 310A. Similarly, probe 303 sensing LL is coupled to the non inverting terminal and the probe 302 sensing RA is coupled to the inverting terminal of the differential amplifier 3106. The differential amplifier 310A amplifies the difference LA-RA to generate lead I ECG signal on path 312A. Similarly, the differential amplifier 310B amplifies the difference LL-RA to generate lead II ECG signal on path 312B.

The analog to digital convertor (ADC) 320A and 320B respectively converts the lead I ECG signal and lead II ECG signal to respective digital bit streams. In one embodiment, asigma-delta modulator is employed for converting the analog lead I and lead II ECG signals to digital lead I and lead II ECG signals. The digitized bit streams from ADC 320A and B are provided on path 323A and B. The digital filter 330A and B respectively filters the bit streams 323A and B to eliminate the noise. For example, noise introduced by the electronic components, electrodes, probes etc are filtered by the digital filter 330A and B.The filtered lead I and lead II ECG signals in the digital domain are provided on path 339A and B respectively.

In one embodiment the lead III ECG signal is generated by subtracting lead I ECG signal from the lead II ECG signal (Lead III=Lead II-Lead I). The subtraction operation may be performed on the analog lead I and lead II ECG signal on the path 312 A and B. Alternatively the subtraction operation may be performed in the digital domain using the digital signals on paths 339A and B.

FIG. 3B is a block diagram illustrating the manner in which CT signal is generated in one embodiment. The block diagram is shown comprising buffers 340A-E, resistors 350A-E, capacitor 355, amplifier 360, ADC 370, filter 380 and memory 390. The block diagram is further described in detail below.

In the block diagram, the buffers 340A-C provide a high impedance coupling to the signals LA, RA, and LL sensed by the probes 301, 302, and 303respectively. The buffers 340A-C may be implemented by employing operational amplifier in the non inverting mode with unity gain. The resistors 350A-C in combination with buffer 340D operate to add the LA, RA, and LL signal to provide summed RA+LA+LL signal on path 354 and 356. The buffer 340E, the capacitor 355 and resistor 350D together operate as low pass filter and provide a reference signal(which is filtered and inverted signal of common mode signals of RA+LA+LL).The reference signal is tied to RL probe to cancel out the common mode noise and also provide a DC bias to the Amplifiers.

The amplifier 360, receives the summed RA+LA+LL signal on its non inverting terminal and the reference signal (tied to RL) on the inverting terminal to generate CT signal on path 367. The ADC 370, converts the analog CT signal on path 367 to digital stream. In one embodiment the ADC 370 is implemented as delta sigma modulator. The CT digital signal is provided on path 378. The filter 380 operates to filter the noise in the CT signal to generate noise free CT signal. The noise free CT signal digital values are stored in the memory 390 (the memory may be an internal memory, Flash memory, RAM, Hard disk, etc.). In one embodiment, the resistors 350A-C are set to 200K Ohms, 350D is set to 100K ohms and 350E is set to 1M ohms. The capacitor 355 is set to 1.5 nanofarad.

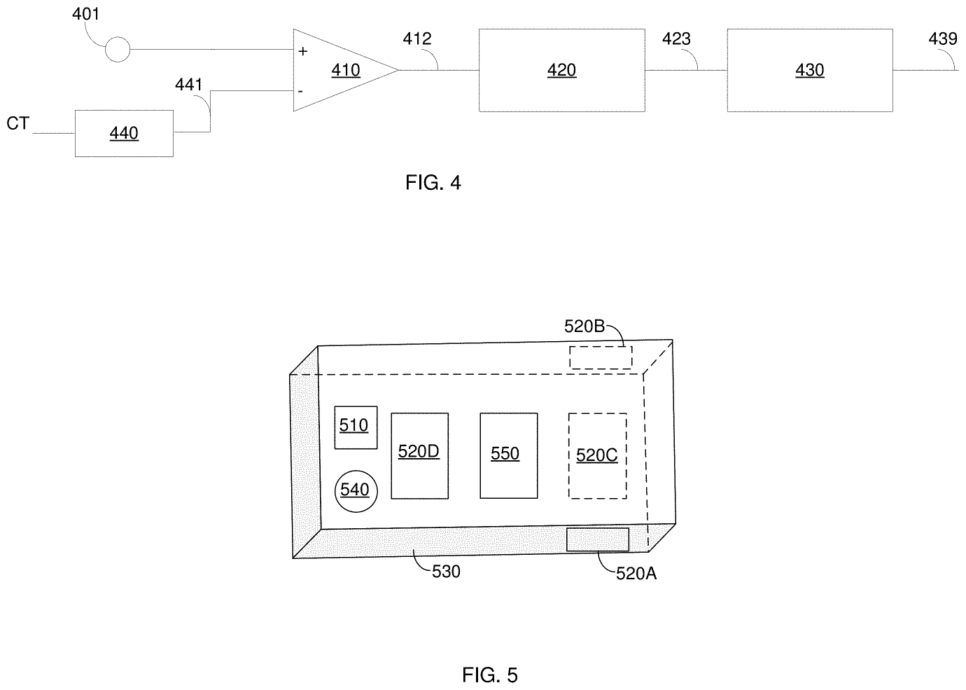

FIG. 4is a block diagram illustrating the manner in which lead V1-V6 signals are generated in one embodiment. The block diagram is shown comprising differential amplifiers 410, analog to digital convertors (ADC) 420, digital filters 430 and digital to analog converter (DAC) 440. In the block diagram, probe 401 is configured to sense electrical signals around chest area at positions V1-V6. The probe 401 is coupled to the non inverting terminal of the differential amplifier 410. The DAC 440converts the CT signal stored in the memory 390 to analog CT signal and provides on the path 441 that is coupled to the inverting terminal of the differential amplifier 410. The differential amplifier 410 amplifies the difference of signal sensed by probe 401 and the analog CT signal on path 441 to generate lead signals V1 through V6 ECG signals on path 412.

The analog to digital convertor (ADC) 420 converts the V1 through V6 ECG signals on path 412 to digital bit streams. In one embodiment, a sigma-delta modulator is employed for converting. The digitized bit streams from ADC 420 are provided on path 423. The digital filter 430 filters the bit streams 423 to eliminate the noise. The filtered V1 through V6 ECG leads signals in the digital domain are provided on path 439. In one embodiment, a timer is employed to indicate the successful detection and conversion of the signal sensed by the probe 301-303, 401 for predetermined time period at their respective position. The corresponding one or more of the 12 lead ECG signal is stored/buffered for presenting the twelve lead ECG signal.

Accordingly, the lead I, II are provided on path 339A and B, the lead III signal is provided by employing a subtractor (not shown) as Lead III=Lead II-Lead I, the CT signal is stored in the memory 390, and the V1-V6 signals are provided on path 432 in sequence as per the positioning of the probe 401. The manner in which leads aVR, aVL and aVF may be generated in the 12 leads ECG signal generator 150 is further described below.

The 12 leads ECG signal generator 150 employ adder, subtractor and divider circuitry to generate the aVR, aVL and aVF in an embodiment. The lead signal aVR is generated through electronic circuitry that perform operation as: aVR=—(lead I+Lead II)/2. Similarly, aVL and aVF are generated using relations: aVL=(lead I-lead II/2) and aVF=(lead II-lead I/2). Thus generating twelve leads ECG signal from four probes (like 101-104 or 301-303 and 401).

The manner in which device 101 is employed for generating the twelve lead ECG signals in en embodiment is further described below.

FIG. 5 is a block diagram of a hand held device generating twelve lead ECG signals in one embodiment. The device 501 is shown comprising stage selector button 510, electrode pads 520A-520D, package 530, beeper 540 and display 550. Each element is further described below.

The electrode pads 520A-520D are shown mounted on the surface of the package 530. The package 530 houses the elements described in sections above. The electrode pads 520A-C are respectively is coupled to the probes 301, 302, and 303. Alternatively, the pads 520A-C may operate as probes 301, 302, and 303. In that, electrode pads 520A and 520B are mounted on the two opposite side surface of the package 530 and the electrode pad 520C is mounted on the bottom side of the package 530. The pad 520D is mounted on the top surface of the package 530 and is coupled to the probe 401. Alternatively, the electrode pad 520D may operate as the probe 401. The selection button 510 enable selection stages and/or switching from one stage to other. Beeper 540 provides audio signal indicating completion of an operation as described in the sections below. The display 550 provides the twelve lead ECG signals and also other guiding indications for operations. The manner in which the device 501 may be operated is further described below

In operation, the button 510 is pressed to select stage one operations. The selection of stage one operation may be confirmed on the display by pressing the button 510. When in the stage one, the user may place the right hand thumb on the pad 520A, left hand thumb on the pad 520B and the bottom pad 520C on the thigh together. The beeper 540 send out a beep signal to indicate successful capturing and generation of lead 1, II, III, aVL, aVR, aVF and storing of CT signal in the memory. After receiving the beeper sound the user may release the thumbs and thigh from the respective pads and operate button 510 again to select the stage two operation. The selection of stage two operation may be confirmed on the display by pressing the button 510.

On successful selection of the stage two operations, user may place device on the chest such that the pad 520D makes contact at position V1. The beeper 540 sends out a beep on successful capturing of the electrical signal from position V1 and generation of V1 lead signal. The user may move/slide the device 501 such that the pad 520D make contact at position V2 to generate the V2 lead signal. In a similar way, on each beep signal, user may slide the device to make contact at V3-V6 positions on successive beeps to generate lead V3-V6 signals. The selection button may be operated to show the result on the display. On pressing the button 530 for display, the device 501 presents twelve leads ECG signals on the display device.

The number of buttons, and beeps and or sound may be employed to provide more varied selection information, positioning information, etc. for example, device 501 may be employed to detect the positioning of the electrode accurately by comparing the signal, signal strength with stored reference signal, or history of the data stored successively. In an yet another alternative, the electrode pad 520D may be made flexibly pulled out of the package 530 to place the pad 520 on the chest while seeing the display. All such extension of the implementation that are apparent to a person skilled in the art by reading this disclosure is covered by this disclosure and the claims.

While various embodiments of the present disclosure have been described above, it should be understood that they have been presented by way of example only, and not limitation. Thus, the breadth and scope of the present disclosure should not be limited by any of the above-discussed embodiments, but should be defined only in accordance with the following claims and their equivalents.

What is claimed is,:

1. A hand held device providing twelve lead ECG signals using fewer number of probes/electrodes comprising:
a first electrode to sense electrical RH (right hand) signal;
a second electrode to sense electrical LH (left hand)signal;
a third electrode to sense electrical LL (left leg) signal;
a fourth electrode to sense a set of electrical V1 through V6 signals in a sequence;
a first electronic circuit operative generate a lead I, lead II and lead III ECG signals;
a second electronic circuit to generate a CT signal from the lead I, lead II and lead Ill signals; and
a third electronic circuit operative to generate a set of V1-V6 lead ECG signals from the set of electrical V1 through V6 signals and the CT signal.

2. The hand held device of claim 1, further comprising an enclosure housing the first electronic circuit, the second electronic circuit and the third electronic circuit, in that, the first electrode, the second electrode, the third electrode and the fourth electrodes are formed as a touch pads on outer surface of the enclosure such that, at least two touch pads are formed on different sides of the enclosure.

3. The hand held device of claim 2, wherein the second electronic circuit is configured to perform operation represented by a relation: CT=(RA+LA+LL)/3 and the CT value is stored in a memory in a digital format.

4. The hand held device of claim 3, wherein the third electronic circuit is configured to perform sequential operation representing the relation: Lead V1 signal=V1-CT, Lead V2 signal=V2-CT, Lead V3 signal=V3-CT, Lead V4 signal=V4-CT, Lead V5 signal=V5-CT, and Lead V6 signal=V6-CT.
